(19) United States
(12) Patent Application Publication
(10) Pub. No.: US 2023/0420122 A1
(43) Pub. Date: Dec. 28, 2023

(54) MONITORING OF ANOMALIES IN BEHAVIOR TO INCREASE QUALITY OF SOFTWARE DEVELOPMENT

(71) Applicant: Kyndryl, Inc., New York, NY (US)

(72) Inventors: Shaleen TONGIA, Bangalore (IN); Kalpesh SHARMA, Bangalore (IN); Soumendra SAHOO, Bhubaneswar (IN)

(21) Appl. No.: 17/847,904

(22) Filed: Jun. 23, 2022

(51) Int. Cl. G16H 40/63

(52) U.S. Cl. CPC G16H 40/63

(57) ABSTRACT

A method includes: identifying, by a computing device, an emotional awareness of an individual based on work product factors; analyzing, by the computing device, an impact on a product development process based on the emotional awareness of the individual; and providing, by the computing device, recommendations to improve the product development process based on the impact.

### BACKGROUND

Aspects of the present invention relate generally to software development and, more particularly, to monitoring of anomalies in behavior to increase quality of software development and deliverables.

Individuals are not always working at their highest level of potential due to many different factors including, for example, emotional health. For example, emotional health may influence or impact the efficiency and hence optimal utilization of an individual when developing software products. Moreover, changing working environments that create, in some instances, isolation due to remote logins, can also influence or impact the efficiency and optimal utilization of an individual when developing software products. These different instances, amongst others, can lead to an inefficient or low quality work product which may later require more active attention either from an individual or a subset of an entire team to meet required expectations of the work product.

### SUMMARY

In a first aspect of the invention, there is a computer-implemented method including: identifying, by a computing device, an emotional awareness of an individual based on work product factors; analyzing, by the computing device, an impact on a product development process based on the emotional awareness of the individual; and providing, by the computing device, recommendations to improve the product development process based on the impact.

In another aspect of the invention, there is a computer program product including one or more computer readable storage media having program instructions collectively stored on the one or more computer readable storage media. The program instructions are executable to: capture data from different development, security, and operations sources; analyze the data to generate a scoring to gauge an outcome of the DevSecOps process; proactively identify problems that require remediation within the DevSecOps process based on the scoring; and provide recommendations for the remediation within the DevSecOps process based on the identified problems.

In another aspect of the invention, there is system including a processor, a computer readable memory, one or more computer readable storage media, and program instructions collectively stored on the one or more computer readable storage media. The program instructions are executable to: ingest data from multiple sources within a development cycle process; determine relevant data for the development cycle process; provide a weight to the relevant data; calculate a score for each of the relevant data using the weight; calculate an overall score for all of the relevant data using the score for each of the relevant data; predict an outcome of the development cycle process using the overall score; provide a recommendation to improve the outcome of deliverables within the development cycle process; and provide a remediation based on the recommendation.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a cloud computing node according to an embodiment of the present invention.

FIG. 2 depicts a cloud computing environment according to an embodiment of the present invention.

FIG. 3 depicts abstraction model layers according to an embodiment of the present invention.

FIG. 4 shows a block diagram of an exemplary environment in accordance with aspects of the invention.

FIG. 5 shows a block diagram of a processing engine, amongst other features, in accordance with aspects of the invention.

FIG. 6 shows a flowchart of an exemplary method in accordance with aspects of the invention.

### DETAILED DESCRIPTION

Aspects of the present invention relate generally to software development and, more particularly, to monitoring of anomalies in behavior to increase quality of software development and deliverables. According to more specific aspects of the invention, methods, systems and computer program products identify patterns related to an emotional state of an individual along with an impact analysis of such well-being, and provides remediations or suggestions to optimize the productivity of the individuals to improve the overall product. In embodiments, the methods, systems and computer program products may evaluate many individual contributors (work product factors) throughout an entire development ecosystem to increase the quality of the product. In this manner, implementations of the invention deliver insights about the state of the individual, how it may impact the overall product development ecosystem and, provide remediation to optimize the productivity of the individual and overall software product.

In specific embodiments, the methods, systems and computer program products capture data from different DevSecOps (i.e., development, security, and operations) sources and analyze the data to generate a scoring mechanism to help gauge an outcome of the DevSecOps process. The score may be used to proactively help users identify problems that require remediation. For example, knowledge about emotional patterns (behavioral anomalies) will be used to drive better decisions on any given day and can strengthen the existing DevSecOps processes to drive awareness about additional/reduced scope of work and hence provide an impetus to the quality and overall productivity of the individual.

Accordingly, the methods, systems and computer program products provide a technical solution to a technical problem. For example, the methods, systems and computer program products ensure high quality and timeliness of software development projects by considering multi-factor dependent individual emotional factors (e.g., work product factors) and establishing patterns that can influence devSecOps. This approach will make the DevSecOps process emotionally aware and make the quality of the deliverables resilient to emotional patterns or other behavioral anomalies. For example, by considering different factors or anomalies in work product, it is possible to gauge deliverable efficiency to determine the influence of such factors on the product or deliverables. This can be perceived in various shapes and formats, e.g., an aggressive day can make a developer introduce more regressions, an engineer that has too many disturbances may cause more functional issues in the code, or hurried execution of QA can lead to additional issues.

It should be understood that, to the extent implementations of the invention collect, store, or employ personal information provided by, or obtained from, individuals (for example, emotional awareness) such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium or media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

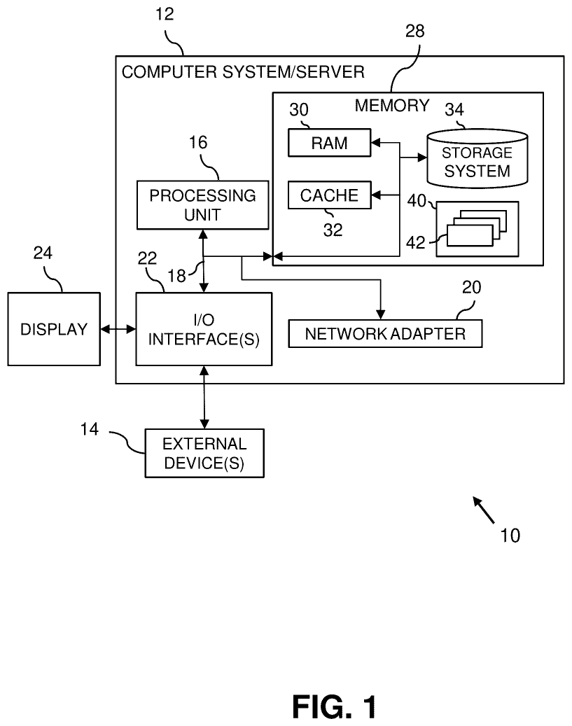

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

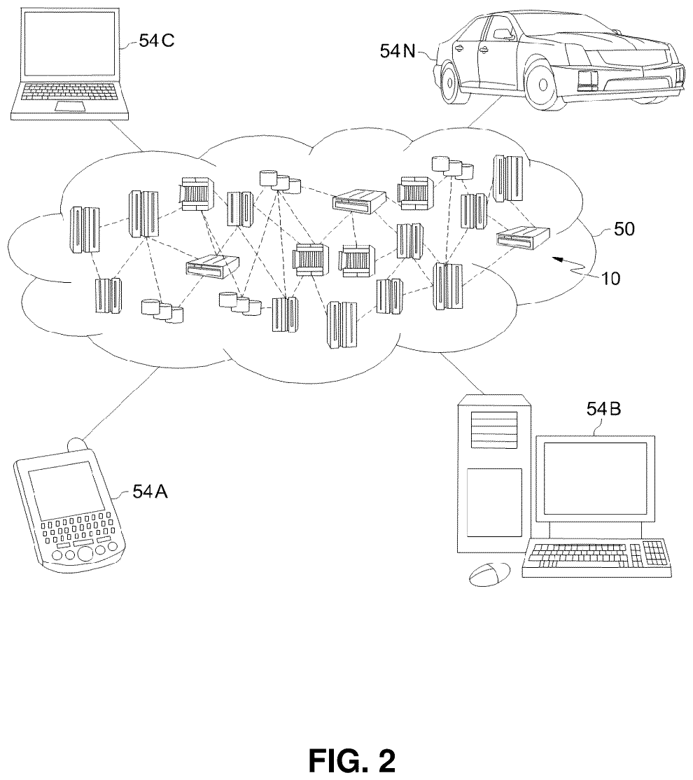

Referring now to FIG. 2, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

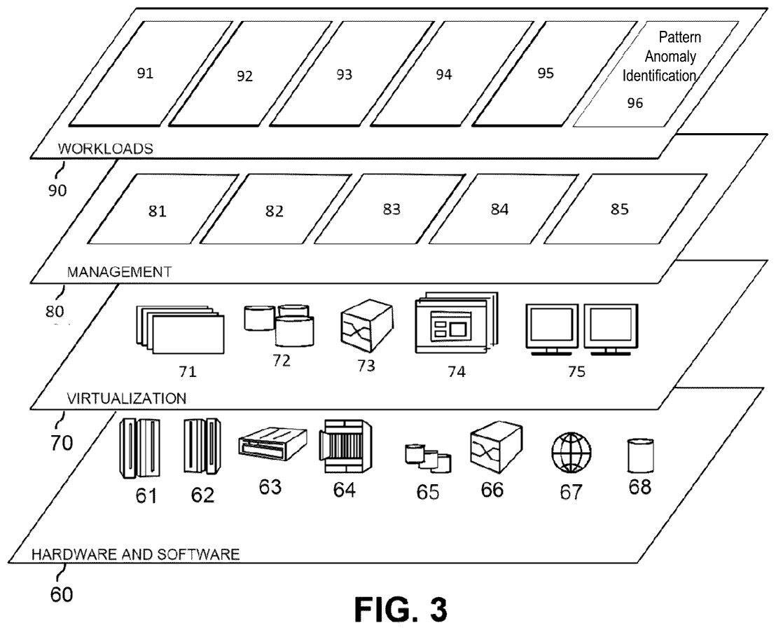

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and pattern or anomaly identification 96.

Implementations of the invention may include a computer system/server 12 of FIG. 1 in which one or more of the program modules 42 are configured to perform (or cause the computer system/server 12 to perform) one of more functions of the pattern or anomaly identification 96 of FIG. 3. For example, the one or more of the program modules 42 may be configured to:
(i) compute an emotional score for each individual or team member to establish patterns or identify anomalies on a day-to-day basis based on a development ecosystem;
(ii) predict defect density in particular functional modules and insights to help align resources to correct such defects;
(iii) determine an impact of the emotional state of an individual or team on software development and planned deliverables. This will make the entire pipeline robust against factors impacting an emotional score and will result into deliverables being more predictable and remain as planned; and
(iv) proactively provide remediation to optimize the productivity of the individuals and overall software product.

As to the latter feature (iv), as software development becomes modernized with new tools and pipelines becoming commonly adopted practices, the proactive remediation can be injected directly into the pipeline to make the entire process resilient against the emotional state of individuals and the team, hence improving quality of deliverables.

Accordingly, the methods, systems and computer program products may be used to identify an emotional state of individuals (e.g., developers, engineers, programmers, etc.) and analyze the impact of these emotional states on the product/software development. The methods, systems and computer program products can use different factors to assess emotional states, and to provide solutions for optimizing productivity of the individuals and increase quality and efficiency of developing the product (software). For example, the methods, systems and computer program products may predict the influence of emotional scoring on deliverables and support better resource alignment to achieve deliverables with improved quality. The methods, systems and computer program products are thus an advanced devOps pipeline that will make the entire process resilient against factors impacting the emotional scoring of the team and will result into deliverables being more predictable and remain as planned.

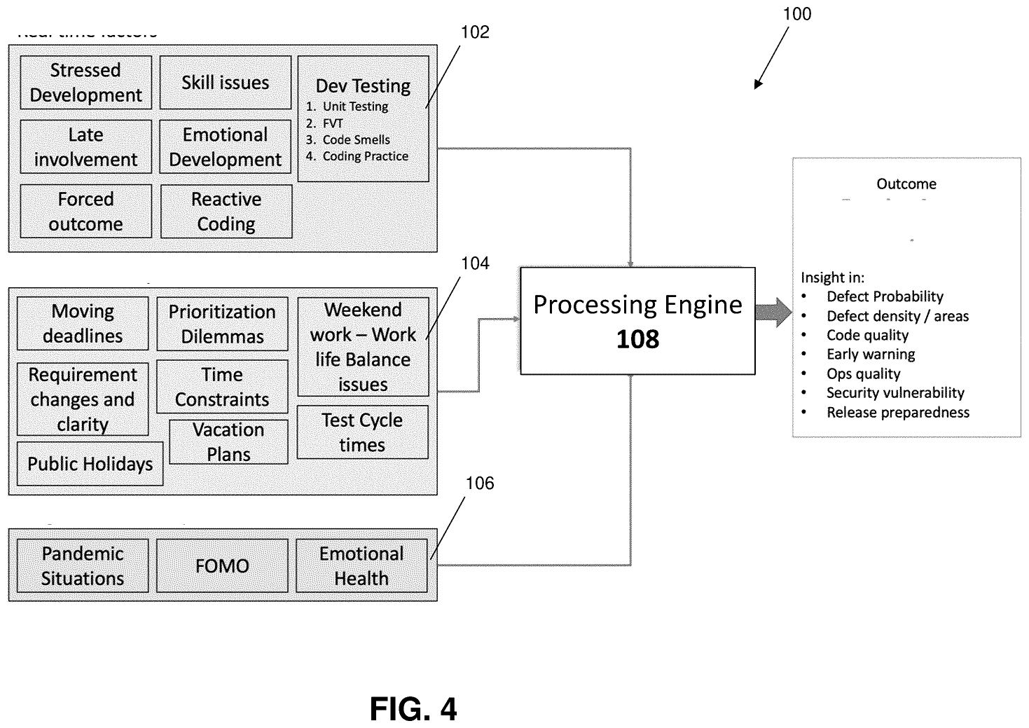

FIG. 4 shows a block diagram of an exemplary environment in accordance with aspects of the invention. In embodiments, the environment 100 includes real-time module 102, a mid-term module 104 and a long-term module 106. The real-time module 102, mid-term module 104 and long-term module 106 ingest or collect data from a wide range of sources for different governing factors in a development pipeline to derive an emotional assessment of the developer/programmer or other team member using processing engine (e.g., DevSecOps processing engine) 108. In embodiments, the entire dataset can be consumed to derive patterns of the individual or team, as a whole, with data already being collected during the development cycle of a software product. By way of example, the following types of data and/or tools can be used to assess the emotional awareness of the individual: stressed development; skills; agile dashboards code; check-in information; static tool analysis reports; vacation planners; requirement updates; development style information; company announcements; test execution results, etc.

More specifically, the real-time module 102 may collect and ingest data that occurs on a daily basis. This data may be associated with the emotional awareness of the developer, programmer, or other team member (hereinafter referred to as an individual), and may be received from different data sources as described below.
(i) Stress development: This refers to factors which may impact the emotional awareness of the individual, for example, based on a daily occurrences such as, e.g., checking coding issues on a daily basis. This may also include issues related to, e.g., coding style, Git Checkin habit, Sonarqube results, time sheets input, change of priorities or requirements, etc. This may be detected by different tools within the current ecosystem such as static coding analysis tools, coding style checking tools as is known in the art (e.g., GITHUB, Sonarqube, Checkstyle, etc). As understood by those of skill in the art, SonarQube is a code quality assurance tool that collects and analyzes source code and provides reports for the code quality. Also, as understood by those of skill in the art, Checkstyle is a development tool to help programmers write Java code that adheres to a coding standard, e.g., number of lines required, syntax, etc. Moreover, as understood by those of skill in the art, Git is software for tracking changes in any set of files, usually used for coordinating work among programmers collaboratively developing source code during software development.
(ii) Late involvement: This refers to factors which may impact the emotional awareness of the individual based on requesting a project be done late in the development cycle and that is to be delivered in the near future (e.g., as detected in an assignment history). This may be detected by a project management tool as is known in the art (e.g., Jira, Agile or other work tracking tool as is known in the art). It should be understood by those of skill in the art that Jira and Agile are a suite of work management solutions (software products) that powers collaboration across teams.
(iii) Forced Outcome: This refers to factors which may impact the emotional awareness of the individual based on a request to complete a project that was not originally planned. This may be detected by a project management tool as is known in the art (e.g., Jira, Agile or other work tracking tool as is known in the art). For example, this may be detected by, e.g., Jira history about the time provided for development to release.

Skill issues: This refers to factors which may impact the emotional awareness of the individual based on certain skill gaps due to, e.g., fast changing technology. This may be detected using a user profile, and using tools such as skill tracking tools, Git contribution history, etc.

Development Testing: This refers to factors which may impact the emotional awareness of the individual based on a process, which kicks off for each checkins by individuals. For example, this type of testing allows monitoring at the very initial signs of code quality of the individual. Code generated by the individual will undergo development level testing using automation testing, static code analysis, unit testing, code coverage, code smells and other checks, which will provide the development level warnings. This information may be detected from GITHUB, Sonarqube, etc.

Emotional Development: This refers to a stress based on knowledge of real-time tracking of individual input. For example, this tracking may include change in quantity of checkin, build failure spike, quantity spike, degradation of code quality, etc. This may be detected by tools such as Travis, Github Actions, Static code analysis spike, etc. As understood by one of skill in the art, Travis is a hosted continuous integration service used to build and test software projects hosted on Github.

Reactive Coding: This refers to factors which may impact the emotional awareness of the individual based on quality of code or build quality. This may be detected by tools such as Travis, Github Actions, Static code analysis spike, etc.

The mid-term module 104 may collect and ingest data that occurs on an intermediate basis (e.g., non-daily basis). This data may be received from different data sources as described below.

Moving Deadlines: This refers to factors which may impact the emotional awareness of the individual based on moved deadlines. This data may be obtained from a history report, e.g., Jira history, and detected by tools such Jira, Agile Management tools and other work tracking tools as is known in the art.

Prioritization Dilemmas: This refers to factors which may impact the emotional awareness of the individual based on prioritization conflicts or changing priorities. This data may be obtained from a history report, e.g., Jira history, and detected by tools such Jira, Agile and other work tracking tools as is known in the art.

Time Constraints: This refers to factors which may impact the emotional awareness of the individual based on time constraints such as deadlines, backlogs, changes in priority, etc. This data may be obtained from a history report, e.g., Jira history, and detected by tools such as Jira, Agile and other work tracking tools as is known in the art.

Requirement Changes and Clarity: This refers to factors which may impact the emotional awareness of the individual based on changes made to deliverables. This data may be obtained from Jira acceptance, description, comments changes, etc. This may be detected by tools such as Jira, Agile and other work tracking tools as is known in the art.

Public Holidays: This refers to factors which may impact the emotional awareness of the individual based on upcoming holidays and the need to finish a project or anticipation of leaving the office for a long weekend, etc. This data may be obtained from a company holiday calendar or other human resource tools.

Vacation Plans: This refers to factors which may impact the emotional awareness of the individual based on upcoming vacation and the need to finish a project or anticipation of leaving the office for an extended time, etc. This data may be obtained from a company vacation calendar or planner or other human resource tools.

Work Life Balance: This refers to factors which may impact the emotional awareness of the individual based on work life balance issues such as working on weekends, holidays, over vacations, etc. This data may be obtained from user time sheets, management requests, weekend updates, etc. This may be detected by tools such as human resource tools, time sheets, calendar applications, etc.

Testing cycle time: This refers to factors which may impact the emotional awareness of the individual based on testing cycles, e.g., whether the code is proper, how much new code has been generated, how much new lines of code have been added, comments added to where code is correct, etc. This data may be obtained by Jira planning board, time allocation history, testing cycle allocation from project management tool data, etc. This may be detected by tools such as Jira, Agile, work tracking tools and QA (quality assurance) automation times.

The long-term module 106 may collect and ingest data that occurs on a longer term basis. This data may be received from different data sources as described below.

FOMO (fear of missing out): This refers to factors which may impact the emotional awareness of the individual based on natural behaviour understanding, users' eagerness, user behaviour, etc. This may be detected by management updates, as an example.

Pandemic Situation: This refers to factors which may impact the emotional awareness of the individual based on a pandemic or other world or local event. This may include working from remote locations, lack of collaborative efforts, etc. This is obtained by company-wide announcements, general news, etc.

Emotional Health: This refers to factors which may impact the emotional awareness of the individual based on user emotional health. This is obtained by using different tools as is known in the art such as daily health trackers or daily mood trackers, as examples.

Corporate Action: This refers to factors which may impact the emotional awareness of the individual based on company news that might affect the individual, e.g., layoffs, reorganizations, etc. This is obtained from company announcements, employee dashboards, etc.

In embodiments, the data from the different modules 102, 104, 106 may be used by the processing engine 108 to develop a score of individual factors and an overall score of multiple factors. Also, the processing engine 108 may use this data to discern patterns in the data collected about the behavioral aspects of the individuals and provide certain outcomes. The outcomes, for example, may include: (i) defect probability, (ii) defect density/areas, (iii) code quality, (iv) early warnings, (v) operations quality, (vi) security vulnerability and/or (vii) release preparedness, amongst other outcomes that can be used to predict quality of the software product and deliverables. These outcomes may then be used to derive weighted scores, as discussed in more detail below, to understand the influence on the deliverable. The score can be used to determine the intrinsic value of a product and proactively provide recommendations or remediation to improve such outcomes.

In further embodiments, the different modules 102, 104, 106 and processing engine 108 may comprise one or more program modules such as program modules 42 described with respect to FIG. 1. The different modules 102, 104, 106 and processing engine 108 may include additional or fewer modules than those shown in FIG. 4. In embodiments, separate modules may be integrated into a single module. Additionally, or alternatively, a single module may be implemented as multiple modules. Moreover, the quantity of devices and/or networks in the environment is not limited to what is shown in FIG. 4. In practice, the environment may include additional devices and/or networks; fewer devices and/or networks; different devices and/or networks; or differently arranged devices and/or networks than illustrated in FIG. 4.

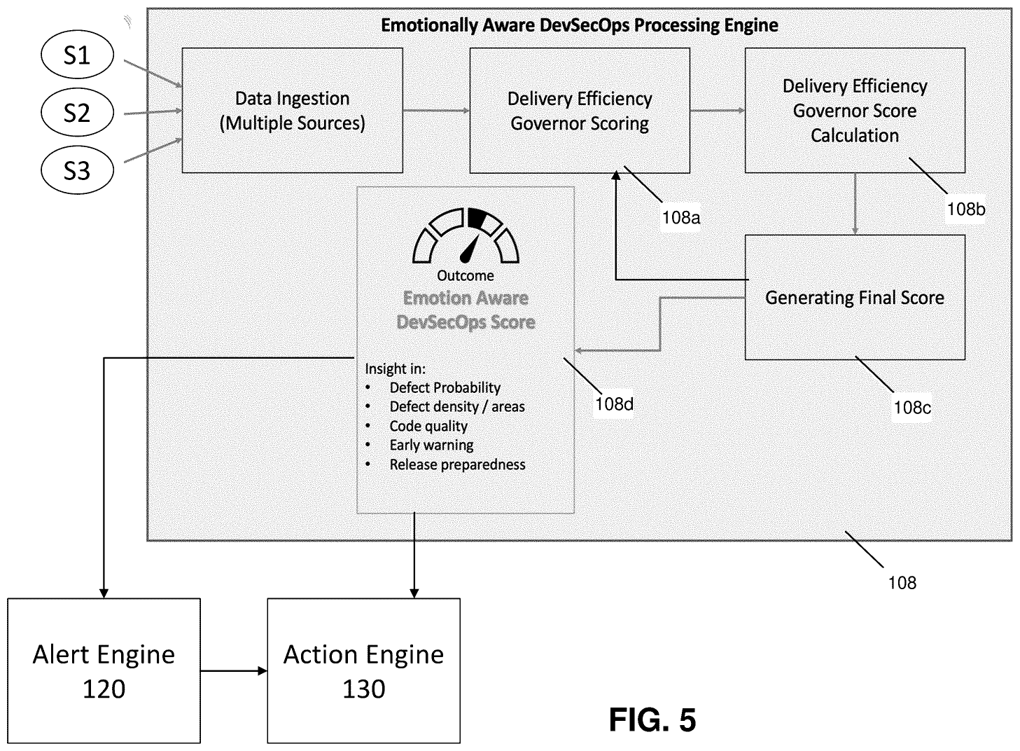

FIG. 5 shows a block diagram of the processing engine 108 in accordance with aspects of the present invention. In embodiments, the processing engine 108 obtains data from different sources, S1, S2, S3. These different sources may be any of the tools within the ecosystem as described with respect to FIG. 4. The data from the different sources may be ingested by the processing engine 108 using different adapters as is known in the art such that no further explanation is required for a complete understanding of the invention.

The processing engine 108 can provide computations of intermediate governing scores from various ingested pipelines (e.g., sources of data). For example, in module 108a, a weight may be provided to any of the particular ingested factors of relevance for a particular product or deliverable such as, e.g., time constraints, priorities, etc. as noted in FIG. 4; whereas, in module 108b, a score can be calculated for the particular factor using the weighting. For example, factors associated with prioritization may be weighted more heavily than factors associated with changes in requirements or sick days taken, etc. In embodiments, a weighting may depend on several factors as noted below. For example, a lower weighting may be used to minimize the importance or completely disregard the factor as not being of any importance for a particular product or deliverable. It should be understood, though, that other weights are also contemplated herein and that the above is merely a non-limiting illustrative example.

It should be understood that the weighting can be learned over a certain time period using an iterative learning process based on a learned importance of such factors for a certain project. By way of illustration, the weights can be determined using artificial intelligence and machine learning to carry out continuous learning and patterns by processing different datasets collected from sources like code repositories, code analysis tools, Agile management tools, automation test results, company dashboards announcements, etc.

More specifically, for example, through an iterative process it may be determined that factors may not be as important as thought, e.g., it does not significantly impact the outcome of the product or deliverable, and, as such, the weight can be lowered. Similarly, if the factor is shown through experience to be important, the weight can be raised. More specifically, in embodiments, the weights may change based on a feedback loop in which the processing engine 108 determines over time that some factors may be more important than other factors for certain projects or deliverables. In this way, the processing engine 108 and, more specifically, the module 108a may include intelligence to determine or ascertain certain requirements and how they may be affected by certain factors, with each of the factors being weighted based on their importance to the outcome, as is learned by the system. Accordingly, with use of the weights, the intermediate scores will help establish the emotional awareness about the development ecosystem, and what problems may be developing or have developed based any of the particular factor which have been ingested and analyzed by the processing engine 108.

In module 108c, a final score can be generated based on the individual scores which have been analyzed by the processing engine 108. This overall score can then be used in module 108d to provide insights into the emotional well-being of the individual and how that may affect outcomes, e.g., defect probability, defect density/area, code quality, early warnings or release preparedness, amongst other examples. For example, the final score may be used to predict poor or undesirable outcomes, hence providing an early warning system to take corrective action to improve the development cycle. Accordingly, the final score can be used to trigger an action to improve an outcome or prevent undesirable outcomes. The final score can also be modified based on a feedback loop back to module 108a, where the weighting can be further refined as described herein.

This score may then be provided to an alert engine 120. The alert engine 120 can provide alerts to the stakeholders, e.g., individual, team or management, of any potential issues that may occur or have occurred based on the emotional state of the individual. The alert can be provided by many different mechanisms including emails, dashboards, text messages or other tools as is known in the art.

The outcome provided in module 108d may also be used in the action engine 130. The action engine 130 may provide recommended courses of action (e.g., remediation) to take in order to alleviate certain stresses and improve overall quality of the product and timeliness of the deliverables, as examples. This may include requesting additional training for the individual, an improved work balance, less conflicts in prioritization, etc. Accordingly, in this way, the systems and methods described herein provide real-time predictive insights on issues with respect to the product such as, e.g., the defect probability code reviews, early warnings recommendations, etc. This, in turn, allows the team or management to provide improved planning processes for the product and deliverables.

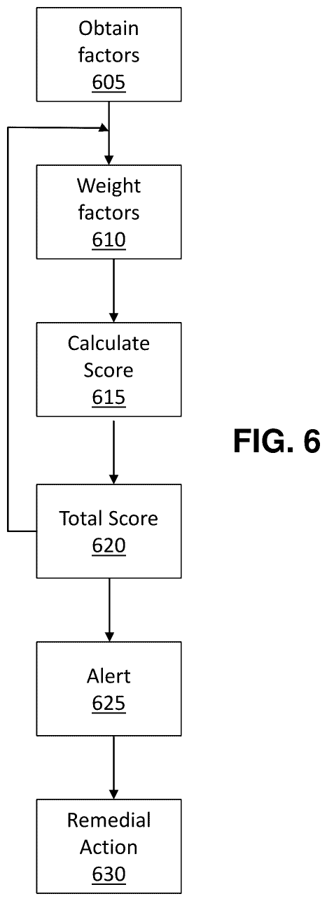

FIG. 6 is a flowchart of an exemplary method in accordance with aspects of the present invention. Steps of the method may be carried out in the environment of FIGS. 4 and/or 5 and are described with reference to elements depicted in FIGS. 4 and/or 5.

At step 605, the system obtains factors which may impact the emotional awareness of the individual. These factors may be ingested from different sources and tools as described with respect to FIG. 4, for example. At step 610, a weight is provided to the different factors. As noted, the weights may vary depending on their importance to a particular project or deliverable. Also, the weight may be adjusted through an iterative learning process in which the weight of a particular factor may be adjusted based on previous experiences as to how such factor affected a certain product or deliverable.

As step 615, a score is calculated for each individual factor using the weighting. In embodiments, the higher the score, the better the emotional stability and, hence, product development is less prone to issues. For example, it may be found that only the following factors may be relevant to a particular product development project in which a score needs to be calculated: vacation days; changing deadlines; checkin history and stressed coding. In this case, each of the factors may be assigned a weight and, from the weight, a score can be calculated as described in more detail below.

At step 620, a total score is calculated by summing all of the individual scores. In embodiments, the final score will trigger an action, i.e., the underlying score provides the details in terms of what needs to be fixed or to act on fixing a certain behavior, etc. In further embodiments, the weights and/or score may change based on a feedback loop in which the processing engine determines over time that some factors may be more important than other factors for certain projects or deliverables, with each of the factors being weighted based on their importance to the outcome, as is learned by the system and shown by the feedback loop.

At step 625, an alert may be provided. In embodiments, the alert may be provided as a proactive measure indicative that an outcome may be negatively affected by the emotional well-being of the individual. At step 630, remediation may be suggested or acted upon to improve the outcome related to the product or deliverable. This remediation can be improved testing, training for the individual, different prioritization schedules, improved coding syntax, etc.

The following is an illustrative use case implementing the systems, methods and computer program products described herein. In this example, the following factors are being utilized: days left before going on a planned vacation; changing deadlines; development anomaly; and stressed coding.

(i) Days Left Before Going on Planned Vacation:
Data Extraction source: Vacation planner.
Variable name (Vd): this is a difference between the time of vacation start to the current date.

Through an iterative learning process, a relation of the planned vacation can be found to be directly proportional to outcomes. For example, the smaller number of days left for vacation may result in a lower score with a higher multiplying factor. In this example, the factor is directly proportionate to the outcome. Also, the effect of vacation is proportional to the closeness with the vacation start date. For example, nearing the vacation start date would influence an individual on the emotional scoring. Also, the significance of an upcoming holiday may be an influencer of emotional well-being, with more significant holidays having a higher affect. Holidays falling on long weekends (planned vacations) may also have a more negative effect on outcomes.

Example: John Doe is planning a vacation in 2 months (e.g., 60 days). Vd1=60. Jack Smith, on the other hand, is planning his vacation in 2 days. Vd2=2. There need be no weights provided in this calculation.

(ii) Changing Deadlines:

Data Extraction source: Jira.

Variable name (Cd): this is a change in deadline (i.e., earlier deadline date—current deadline date).

Through an iterative learning process it has been found that the higher the change, the less the score. The factor depends on how far away the new deadline is from the current date. This factor is inversely proportional to the outcome.

Example: John Doe has almost no change in deadline (same dates). Cd1=100 (no difference), where 100 is a constant to keep all the values in same scale. Jack Smith, on the other hand, has aggressive changes in the deadline. In this case, the current deadline is reduced by 10 days from 20 days, hence an approximate 50% reduction. The 50% reduction may be the weight and afforded a value of, e.g., 2. Accordingly, the overall score Cd2=100/2(50%)=50. In this example, John Doe has a higher score and, hence, outcomes for John Doe would be better than Jack Smith.

(iii) Development Anomaly:

Data Extraction source: git checkin history.

Variable name (GQ): this is the quantity of git checkin in the repository.

Related factors may be new work added to the team (JIRA can confirm the assignments to user), last minute check in before release (release dates extracted from JIRA), amount of defect fixes (defect count extracted from JIRA), and/or code quality difference (code quality checks from Sonarqube).

Through an iterative learning process is has been found that the higher the anomaly, the lower the score value which will eventually drop the overall score. For example, the lesser the anomaly, the higher the code quality.

Example: John Doe has a very minimal anomaly, with no change in the regular check in pattern and quality. Accordingly, GQ1=100 (no anomaly). Jack Smith, on the hand, has increased new check ins (e.g., 50% increase from regular time), a defect from QA automation shows an increase in failures of 25%, and defect fixes have increased by 50%. According, weights of 50% (value of, e.g., 2), 25% (value of, e.g., 1) and 50% (value of, e.g., 2) are provided, with the overall score of 20, e.g., GQ2=20 (e.g., 100/(2(50%)+1(25%)+2(50%)) or 100/5. In this example, John Doe has a higher score and, hence, outcomes for John Doe would be better than Jack Smith.

Stressed Coding:

Data Extraction source: JIRA and Git

Variable name (SQ): this refers to code checkins, JIRA status and Skill Matrix.

Related factors include, e.g., skill issues (SM) (received from Git skills or Skill Matrix): weekend checkins (LB) (received from Git checkin dates), and late assignment to the work or delivery date or date assigned (LA).

Through an iterative learning process is has been found that higher skill gaps will have more adverse effects on the development process. Also, higher weekend checkin values will have a negative effect on the outcomes, e.g., more defects, etc. Also, a smaller difference of assignment will have a higher impact on outcomes. The factor is inversely proportional and denotes aggressive timelines.

Example: John Doe has the required skills for development and does not have any anomalies or change in assignment. Accordingly, SQ=100. Jack Smith, on the other hand, is lacking the required skills in the project (efficiency reduced to 50%), and due to the lack of skills will be adding extra hours on the weekend (e.g., low quality of product and stressed development, e.g., 50% efficiency). Also, Jack Smith has joined the project recently (25% reduced efficiency). According, weights of 50% (value of, e.g., 2), 25% (value of, e.g., 1) and 50% (value of, e.g., 2) have been accorded, with the overall score SQ=20 (e.g., 100/(2(50%)+2(50%)+1(25%)) or 100/5=20. In this example, John Doe has a higher score and, hence, outcomes for John Doe would be better than Jack Smith.

In these examples, John Doe would have an overall score of 360. Specifically, John Doe's score would be 360, e.g., Score1=Vd1+Cd1+GQ1+SQ1 or Score1=60+100+100+100=360. On the other hand, Jack Smith would have an overall score of 92. Specifically, Jack Smith would have a score: Score2=Vd2+Cd2+GQ2+SQ2 or Score 2=2+50+20+20=92.

In this example, John Doe has a higher score and, hence, overall outcomes for John Doe would be better than Jack Smith. Accordingly, from the scores above, it is now possible to identify that the output generated by Jack Smith may have quality concerns and that the work product may requires increased or better validations. This can be used by a planning team to work on the quality verification for the development generated by Jack Smith and for the need of more validations. This will also signify that there may be a need of overlapping resources or additional resources to be included for work of Jack Smith. This can also provide insight into the following areas for remediation:
Defect probability, i.e., which area of application needs more concentration from the QA team in the current release;
Code Reviews, i.e., where more concentration is needed on the code quality and code verifications;
Early warnings, i.e., which areas are more prone for errors; and
Planning recommendations, i.e., change resource allocation, double up resource allocation, release date planning, feature release decisions and others.

In embodiments, a service provider could offer to perform the processes described herein. In this case, the service provider can create, maintain, deploy, support, etc., the computer infrastructure that performs the process steps of the invention for one or more customers. These customers may be, for example, a business that provides software development. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system/server 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system/server 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
identifying, by a computing device, an emotional awareness of an individual based on work product factors;
analyzing, by the computing device, an impact on a product development process based on the emotional awareness of the individual; and
providing, by the computing device, recommendations to improve the product development process based on the impact.

2. The method of claim 1, wherein the identifying is performed by tools within a development security and operations process ecosystem.

3. The method of claim 1, wherein the analyzing comprises establishing patterns of predetermined work product factors which account for anomalies on a day-to-day-basis based on a defined development process ecosystem.

4. The method of claim 1, wherein the analyzing comprises providing a weight to each work product factor associated with the emotional awareness of the individual and relevant to a particular product development process.

5. The method of claim 4, wherein the weight is determined based on an iterative, learning process which changes over time.

6. The method of claim 1, wherein the work product factors comprise daily factors, intermediate factors and long-term factors.

7. The method of claim 1, wherein the work product factors are each individually scored with a weight applied thereto.

8. The method of claim 7, wherein the individual scores are summed up to provide a final score indicative of the impact on the product development process.

9. The method of claim 8, wherein the final score predicts an influence of the quality and timeliness of deliverables in the product development process.

10. The method of claim 8, wherein the recommendations comprise remediation including resource alignment to achieve deliverables with an increased quality including defect probability, code reviews, early warnings and planning recommendations.

11. The method of claim 1, wherein the computing device includes software provided as a service in a cloud environment.

12. A computer program product comprising one or more computer readable storage media having program instructions collectively stored on the one or more computer readable storage media, the program instructions executable to:
capture data from different development, security, and operations sources;
analyze the data to generate a scoring to gauge an outcome of the DevSecOps process;
proactively identify problems that require remediation within the DevSecOps process based on the scoring; and
provide recommendations for the remediation within the DevSecOps process based on the identified problems.

13. The computer program product of claim 12, wherein the data comprise work product related to emotional well-being of an individual while developing a software deliverable.

14. The computer program product of claim 12, wherein the scoring comprises a weight that is iteratively learned and changed over time based on its importance to the identified problem.

15. The computer program product of claim 12, wherein the scoring comprises a score for individual factors and a sum of the individual factors.

16. The computer program product of claim 15, wherein the scoring predicts an influence of emotional well-being on deliverables and supports improved resource alignment to achieve timely deliverables with improved quality.

17. The computer program product of claim 15, wherein the data is ingested on a day-to-day basis from multiple sources during a development cycle of a software product to assess emotional awareness of an individual working within the DevSecOps process.

18. The computer program product of claim 15, wherein the proactively identify problems comprises at least one of (i) defect probability, (ii) defect density/areas, (iii) code quality, (iv) early warnings, (v) operations quality, (vi) security vulnerability and (vii) release preparedness.

19. A system comprising:
a processor, a computer readable memory, one or more computer readable storage media, and program instructions collectively stored on the one or more computer readable storage media, the program instructions executable to:
ingest data from multiple sources within a development cycle process;
determine relevant data for the development cycle process;
provide a weight to the relevant data;
calculate a score for each of the relevant data using the weight;
calculate an overall score for all of the relevant data using the score for each of the relevant data;
predict an outcome of the development cycle process using the overall score;
provide a recommendation to improve the outcome of deliverables within the development cycle process; and
provide a remediation based on the recommendation.

20. The system of claim 19, wherein the data is associated with an emotional awareness of an individual working on deliverables within the development cycle process.
